(19) United States
(12) Patent Application Publication
(10) Pub. No.: US 2020/0406019 A1
(43) Pub. Date: Dec. 31, 2020

(54) MULTI-LAYER HOSE FOR AN INFUSTION SET FOR DISPENSING A FLUID

(71) Applicant: Ypsomed AG, Burgdorf (CH)

(72) Inventors: Rolf Marggi, Bern (CH); Christoph Renggli, Grenchen (CH); Andreas Zurflüh, Hasle-Rüegsau (CH)

(21) Appl. No.: 17/019,726

(22) Filed: Sep. 14, 2020

Related U.S. Application Data
(63) Continuation of application No. PCT/IB2019/052422, filed on Mar. 26, 2019.

(30) Foreign Application Priority Data
Mar. 29, 2018 (CH) 00419/18

(51) Int. Cl. A61M 39/08; B32B 1/08; B32B 27/40; B32B 27/32

(52) U.S. Cl. CPC A61M 39/08; B32B 1/08; B32B 2597/00; B32B 27/32; B32B 2535/00; B32B 27/40

(57) ABSTRACT

A multi-layer tube for an infusion set for dispensing a fluid includes an outer layer formed of polyurethane and an inner layer formed of high-density polyethylene and/or polypropylene.

### RELATED APPLICATIONS

This application is a continuation of International Patent Application No. PCT/IB2019/052422, filed Mar. 26, 2019, entitled “MULTI-LAYER HOSE FOR AN INFUSION SET FOR DISPENSING A FLUID,” which in turn claims priority to Swiss Patent Application No. 00419/18, filed Mar. 29, 2018, entitled “MULTI-LAYER HOSE FOR AN INFUSION SET FOR DISPENSING A FLUID”, each of which is incorporated by reference herein, in their entirety and for all purposes.

### TECHNICAL FIELD

The invention concerns a multilayer tube, in particular a multilayer tube constructed of polymers. Such tubes are, for example, used in connection with an infusion set in order to dispense or administer a fluid, in particular a fluid medication, to a patient.

### BACKGROUND

Infusion sets can serve to administer fluids, in particular, fluid medications. The infusion set can preferably be connected to a fluid dispensing apparatus, where the fluid, in particular, the liquid medication, can be administered into the body of the patient via a transdermal cannula provided in the infusion set. After use, the infusion set can be replaced by a new infusion set, so that the fluid dispensing apparatus can be used for an additional use.

Tubes or fluid conduits for an infusion set serve to transport the fluid, in particular, the fluid medication, from the fluid dispensing apparatus to the cannula of the infusion set to the patient. Such tubes must meet a number of requirements, in particular, they must exhibit flexibility, guarantee a resistance to tearing or a tensile strength, be compatible with the fluid being dispensed to the patient, and/or guarantee visibility of the phase boundary between a gas, in particular air, and a liquid, in particular a fluid.

The term “medication” here includes any flowable medicinal formulation that is suitable for controlled administration through, for example, a cannula or hollow needle. The medication may be in the form of a liquid, a solution, a gel, or a fine suspension that contains one or more medicinal agents. The medication can be a composition having a single active agent or a premixed or co-formulated composition containing a plurality of active agents from a single container. Medication comprises drugs like peptides (for example, insulins, insulin-containing medications, GLP-1-containing preparations, as well as derived or analogous preparations), proteins and hormones, bio-obtained or active agents, active agents based on hormones or genes, nutrient formulations, enzymes, and other substances, both in solid (suspended) or liquid form, or even polysaccharides, vaccines, DNA, or RNA, or oligonucleotides, antibodies, or moieties of antibodies, and suitable base substances, auxiliary substances, and carriers.

Multilayer tubes for an infusion set are known from the prior art.

A multilayer tube for an infusion set is known, for example, from WO 2013/109329 A1, which describes a three-layer polymer tube for an infusion set having an outer layer of polyurethane, an inner layer of polyethylene, and an intermediate layer of ethylene/ethyl acrylate copolymer, or anhydride grafted ethylene/methyl acrylate copolymer, a copolymer of one or more of said acrylates or a blend thereof. A disadvantage is that the layer between the outer and the inner layer can contaminate the fluid being administered to the patients. It is further disadvantageous that the inner layer of the tube can absorb preservatives from the fluid being administered to the patient, thereby limiting the stability of the fluid being administered.

Thus, it is an objective of the present disclosure to provide a multilayer tube for an infusion set, a method for producing a multilayer tube for an infusion set, and a use of a multilayer tube for an infusion set, where the danger of contamination of the fluid being administered to the patient is reduced. Furthermore, it is an objective of the present disclosure to provide a multilayer tube for an infusion set, a method for producing a multilayer tube for an infusion set, and a use of a multilayer tube for an infusion set, to thereby improve the stability of the fluid being administered to the patient.

### SUMMARY

These aforementioned objectives are reached according to the present disclosure.

Implementations are broadly directed to a multilayer tube for an infusion set. Such tubes can be used in combination with infusion sets in order to administer or dispense a fluid continuously or repeatedly to a patient. Infusion sets are often used in diabetes therapy, where insulin is continuously or repeatedly dispensed to the patient. Multilayer tubes, in particular, multilayer tubes for infusion sets, can also be used for other types of therapy, where a continuous or repeated dispensing of a fluid is to be ensured.

A first end of the tube of the infusion set can have an adapter in order to establish a separable connection to a fluid dispensing apparatus, such as an infusion pump. Furthermore, a piercing cannula can be disposed on the adapter in order to establish a fluid connection to the dispensing apparatus, such as the infusion pump.

The second end of the tube of the infusion set can have a cap with a puncture cannula, wherein the puncture cannula can puncture a septum of a base of the infusion set in order to make a fluid connection between the tube and the base. The tube with the attached adapter and the attached cap can form a part of the infusion set.

The base of the infusion set comprises an adhesive layer, which can be affixed or adhered to the skin of a patient, and a cannula in order to conduct the fluid from the dispensing apparatus, such as from the infusion pump, through the tube and through the cannula into the body of the patient. The base may likewise form a part of the infusion set.

Alternatively, the multilayer tube according to the invention may also be connected, separably or inseparably, to a fluid dispensing apparatus, such as an infusion pump, using different means and/or can be connected to the patient using different means, in order to deliver the fluid to the body of the patient.

Implementations of the present disclosure provide a multilayer tube or hose for an infusion set for dispensing a fluid. A cross-section of the multilayer tube may be round or oval. Alternatively, the inner and/or outer surface of the multilayer tube can have one or more flutes or corrugations. The multilayer tube may have an outer and an inner layer. The outer layer comprises a polyurethane (PU) and the inner layer comprises a high-density polyethylene (HDPE) and/or a polypropylene (PP). In an alternative form of the invention, the multilayer tube can comprise additional layers, chemical or biological compounds, or chemical or biological compounds in such one or more additional layers. For example, the tube may release preservatives, stabilizers, anti-inflammatory substances, or another active agent into the fluid flowing in the tube.

The multilayer tube may comprise or consist of two layers, such as only two layers. A bond may be established between each of the respective layers, such as between the two layers, at their contact surfaces during a coextrusion process via a chemical and/or physical operation. An outer and the inner layer can be joined to each other via a form fit, a force fit, and/or a chemical bond. For example, the outer and the inner layer of the multilayer tube may be joined and/or affixed to each other via a shrink fit process. The two-layer tube may be manufactured through a coextrusion process, and after the tube is coextruded, the tube may, for example, be cooled in a water bath, and may be rolled on a roller and cut to the appropriate length. The two-layer tube may be free of an adhesive layer between the outer and the inner layer, and the outer layer may be a polyurethane (PE), and the inner layer may be a high-density polyethylene (HDPE) or a polypropylene (PP). Omitting an adhesive layer or other intermediate layer between the two layers prevents contamination of the fluid being dispensed to the patient by substances released from the adhesive layer. Moreover, the multilayer tube may be transparent or clear, so that the flow of the fluid or a possible inclusion of air bubbles can be detected, in particular, visually detected.

The outer layer of the multilayer tube comprises a polyurethane (PU), such as a thermoplastic polyurethane (PU), including a thermoplastic polyether polyurethane. The polyurethane (PU) should have good resistance to tearing or good tensile strength, resistance to hydrolysis, cold flexibility, and/or resistance to microorganisms. In some implementations, the outer layer of the multilayer tube is Elastollan® 1185A 10 FC from BASF Polyurethanes GmbH.

The inner layer of the multilayer tube comprises a high-density polyethylene (HDPE) and/or a polypropylene (PP). The high-density polyethylene (HDPE) and the polypropylene (PP) that are used should be compatible with the fluid being dispensed to the patient, so that, for example, no or only low amounts of undesirable chemical/physical reactions and/or interactions take place between the fluid dispensed to the patient and the inner layer of the multilayer tube. For instance, the inner layer of the multilayer tube is SABIC® HDPE B5429 from Sabic Europe and/or polypropylene Bormed™ SC820 CF-11 from Borealis AG.

Furthermore, the multilayer tube according to the present disclosure may be used for an infusion set for diabetes therapy. In this case, a medication, for example insulin, can be delivered to the body of the patient through an infusion pump and through the tube. The insulin being dispensed to the patient is a hormone that comprises amino acids, wherein additional substances are added to stabilize the insulin. These are various preservatives or stabilizers such as, for example, phenol and/or cresol, in particular m-cresol. The stability of the insulin is improved through better preservation of the insulin. Through the use of preservatives in the insulin, the insulin is protected against spoilage, especially microbial spoilage. Since the insulin flows through the tube of the infusion set during delivery from the infusion pump into the body of the patient, the tube should absorb as little preservative as possible from the insulin, so that the stability of the insulin being dispensed to the patient is improved. The absorption of the preservative from the insulin is reduced, and consequently the efficacy of the insulin is improved through the use of a high-density polyethylene (HDPE) and/or a polypropylene (PP) as the inner layer of the multilayer tube.

Furthermore, by adjusting the wall thickness and the inside diameter of the inner layer of the multilayer tube, the volume or the surface area of the inner layer can be reduced so that a smaller volume of inner layer is used to make the tube. By having a smaller volume of the inner layer and a lower surface of contact with the medication, less preservative, in particular phenol and/or cresol, in particular m-cresol, is absorbed from the insulin by the inner layer of the tube. This leads to better stability of the insulin being dispensed to the patient.

In one embodiment, the inside diameter of the inner layer is greater than or equal to 0.22 mm and less than or equal to 0.38 mm, or may be between 0.26 mm and 0.34 mm, or between 0.28 mm and a 0.32 mm, or about 0.30 mm.

In other embodiments, the inside diameter of the inner layer is greater than or equal to 0.03 mm and less than or equal to 0.27 mm, and may be between 0.07 mm and 0.27 mm, or between 0.11 mm and 0.19 mm, or about 0.15 mm.

In an alternative embodiment, the inside diameter of the inner layer can be greater than or equal to 0.42 mm and less than or equal to 0.58 mm, and may be between 0.46 mm and 0.54 mm, or between 0.48 mm and 0.52 mm, or about 0.50 mm.

In another embodiment, the outside diameter of the inner layer can be greater than or equal to 0.5 mm and less than or equal to 0.70 mm, may be between 0.55 mm and 0.65 mm, or between 0.58 mm and 0.62 mm, or about 0.6 mm. Furthermore, the inside diameter of the outer layer can be greater than or equal to 0.5 mm and less than or equal to 0.70 mm, and may be between 0.55 mm and 0.65 mm, or between 0.58 mm and 0.62 mm, or about 0.60 mm. The outside diameter of the outer layer may be greater than or equal to 1.42 mm and less than or equal to 1.62 mm, and may be between 1.47 mm and 1.57 mm, or between 1.50 mm and 1.54 mm, or about 1.52 mm. In an alternative embodiment, the outside diameter of the outer layer may be greater than or equal to 1 mm and less than or equal to 3 mm, such as between 1.30 mm and 2.70 mm, or between 1.60 mm and 2.40 mm, or about 2.00 mm.

In an alternative embodiment, the outside diameter of the outer layer may be greater than or equal to 0.70 mm and less than or equal to 0.9 mm, and may be between 0.75 mm and 0.85 mm, or between 0.78 mm and 0.82 mm, or about 0.80 mm. Furthermore, the inside diameter of the outer layer can be greater than or equal to 0.70 mm and less than or equal to 0.90 mm, and may be between 0.75 mm and 0.85 mm, or between 0.78 mm and 0.82 mm, or about 0.80 mm. The outside diameter of the outer layer may be greater than or equal to 1.42 mm and less than or equal to 1.62 mm, and may be between 1.47 mm and 1.57 mm, or between 1.50 mm and 1.54 mm, or about 1.52 mm. In an alternative embodiment, the outside diameter of the outer layer may be greater than or equal to 1 mm and less than or equal to 3 mm, and may be between 1.30 mm and 2.70 mm, or between 1.60 mm and 2.40 mm, or about 2.00 mm.

### BRIEF DESCRIPTION OF THE DRAWINGS

FIG. 1 shows a cross-sectional view of a tube, according to a first embodiment of the present disclosure.

FIG. 2 shows a cross-sectional view of a tube, according to a second embodiment of the present disclosure.

FIG. 3 shows a lengthwise section of an infusion set comprising the tube according to the first and second embodiments of the present disclosure.

### DETAILED DESCRIPTION

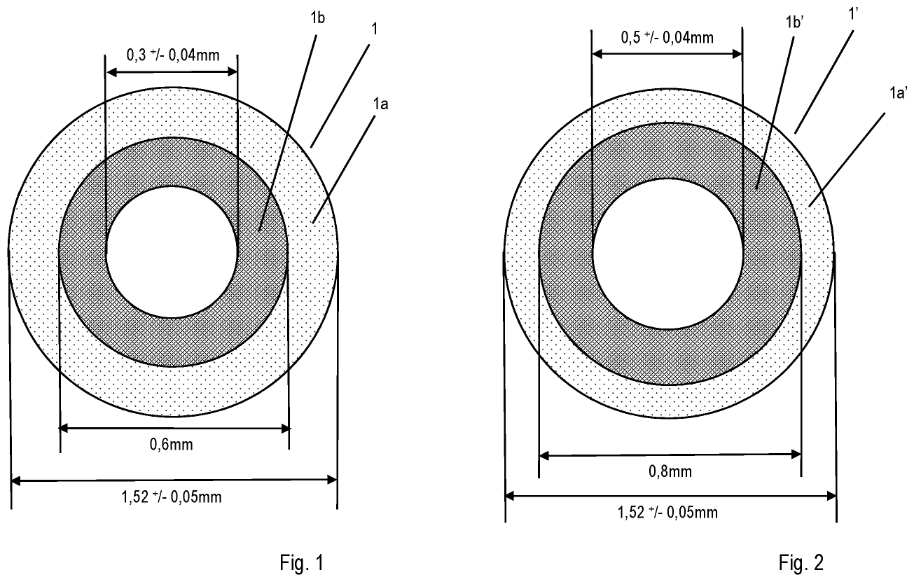

A cross-sectional view of a tube (1) according to a first embodiment of the present disclosure is illustrated in FIG. 1.

The multilayer tube (1) may be for an infusion set for dispensing a fluid, and includes an outer layer (1a) formed of a polyurethane (PU) and an inner layer (1b) formed of a high-density polyethylene (HDPE) and/or a polypropylene (PP). The multilayer tube (1) may consist of only two layers, namely the outer (1a) and the inner (1b) layers. The outer layer (1a) of the two-layer tube (1) may be formed of Elastollan® from BASF Polyurethanes GmbH. The inner layer (1b) of the two-layer tube (1) may be formed of SABIC® HDPE B5429 from Sabic Europe and/or Bormed™ SC820CF-11 polypropylene from Borealis AG. The outer layer and the inner layer may thus each consist of one type of material. In some implementations, the tube (1) may be free of an intermediate layer, such as an intermediate layer of ethylene/ethyl acrylate copolymer, or anhydride grafted ethylene/methyl acrylate copolymer, a copolymer of one or more of said acrylates or a blend thereof, as disclosed in WO 2013/109329 A1 and described herein.

The bond between the outer (1a) and the inner (1b) layers of the two-layer tube (1) may be established during a coextrusion process through a chemical and/or a physical operation. The outer (1a) and the inner (1b) layer may be joined together via a form fit, a force fit, and/or a chemical bond. For example, the outer (1a) and the inner (1b) layers of the multilayer tube (1) may be joined together through a shrink fit process, where the outer surface of the inner layer is pressed against the inner surface of the outer layer during the production process, thus forming a bond, for example through a change of temperature, a change of shape, or relaxation. The cross section of the multilayer tube (1) may be round or oval. Alternative embodiments of the cross-section are also possible.

The outside diameter of the outer material may be about 1.52±0.05 mm and the inside diameter of the outer material may be about 0.60 mm. The outside diameter of the inner material may be about 0.60 mm and the inside diameter of the inner material is about 0.30±0.04 mm.

Through the use of the outside and/or inside diameter of the inner layer (1b), less preservative, in particular phenol and/or cresol, in particular m-cresol, is absorbed by the inner layer of the tube from the fluid, in particular from the insulin. This leads to improved stability of the insulin being dispensed to the patient.

Moreover, through the use of the outside and/or inside diameters of the outer layer (1a), the tube may be resistant to tearing, may have a high tensile strength, may be resistant to hydrolysis, have cold flexibility, and/or be resistant to microorganisms.

A cross-sectional view of a tube (1′) according to a second embodiment of the present disclosure is illustrated in FIG. 2. The second embodiment differs from the first embodiment by the dimensions of the outside diameter and/or the inside diameter of the outer (1a′) and/or inner (1b′) layer of the tube (1′).

The outside diameter of the outer material (1a′) is about 1.52±0.05 mm and the inside diameter of the outer material (1a′) is about 0.80 mm. Furthermore, the outside diameter of the inner material (1b′) is about 0.80 mm and the inside diameter of the inner material (1b′) is about 0.50±0.04 mm.

The advantages previously noted in the first embodiment are achieved through the use of said outside and/or inside diameters of the outer (1a′) and the inner (1b′) layers.

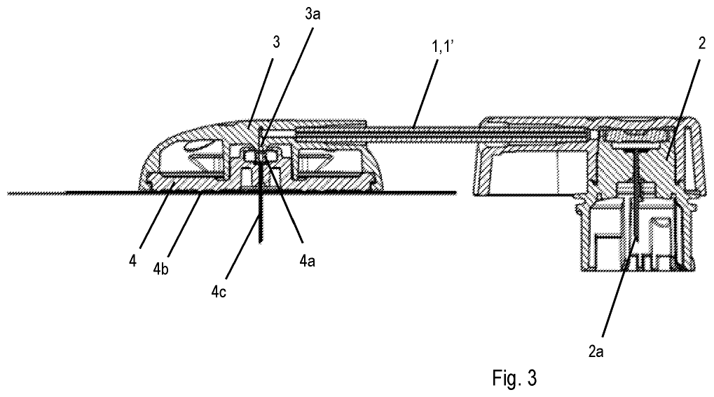

The tube (1, 1′) according to embodiments of the disclosure may be used in connection with an infusion set for dispensing the fluid. An infusion set with a tube (1, 1′) according to embodiments of the present disclosure is shown in FIG. 3. The tube (1, 1′) may alternatively be used with other devices where a transport of a fluid is required. The first end of the tube (1, 1′) may include an adapter (2). The adapter (2) serves to establish a separable or detachable connection or coupling with a dispensing apparatus for dispensing a fluid. The adapter (2) may comprise a piercing cannula (2a) in order to form a fluid connection to the dispensing apparatus, in particular the infusion pump. At the second end of the tube (1, 1′) of the infusion set there can be a cap (3), wherein the cap (3) has a puncture cannula (3a). The puncture cannula (3a) can puncture a septum (4a) of a base (4) of the infusion set in order to form a fluid connection. The base (4) of the infusion set furthermore comprises an adhesive layer (4b), which can be affixed or adhered to the skin of the patient, and a cannula (4c) in order to administer the fluid from the infusion pump through the tube (1, 1′) and through the cannula into the body of the patient. Such infusion sets may be used in connection with diabetes therapy, in particular to dispense insulin. The infusion set with the tube (1, 1′) according to the invention can alternatively be used in connection with other therapies. In diabetes therapy, an infusion pump can be used as the dispensing apparatus, wherein a continuous or repeated dispensing of insulin can be ensured.

### REFERENCE NUMBERS

1, 1′ multilayer, in particular two-layer tube
1a, 1a′ outer layer
1b, 1b′ inner layer
2 adapter
2a piercing cannula
3 cap
3a puncture cannula
4 base
4a septum
4b adhesive layer (plaster)
4c cannula

What is claimed is:

1. A multilayer tube for an infusion set for dispensing a fluid, comprising:
an outer layer comprising polyurethane; and
an inner layer comprising high-density polyethylene and/or polypropylene.

2. The multilayer tube of claim 1, wherein the multilayer tube consists of two layers.

3. The multilayer tube of claim 1, wherein an outside diameter of the outer layer is greater than or equal to 1.42 mm and less than or equal to 1.62 mm.

4. The multilayer tube of claim 3, wherein the outside diameter of the outer layer is between 1.47 mm and 1.57 mm, between 1.50 mm at 1.54 mm, or about 1.52 mm.

5. The multilayer tube of claim 3, wherein an inside diameter of the outer layer is greater than or equal to 0.50 mm and less than or equal to 0.70 mm.

6. The multilayer tube of claim 5, wherein the inside diameter of the outer layer is between 0.55 mm and 0.65 mm, between 0.58 mm and 0.62 mm, or about 0.60 mm.

7. The multilayer tube of claim 3, wherein an inside diameter of the outer layer is greater than or equal to 0.70 mm and less than or equal to 0.90 mm.

8. The multilayer tube of claim 7, wherein the inside diameter of the outer layer is between 0.75 mm and 0.85 mm, between 0.78 mm and 0.82 mm, or about 0.80 mm.

9. The multilayer tube of claim 3, wherein an outside diameter of the inner layer is greater than or equal to 0.50 mm and less than or equal to 0.70 mm.

10. The multilayer tube of claim 3, wherein an outside diameter of the inner layer is greater than or equal to 0.70 mm and less than or equal to 0.90 mm.

11. The multilayer tube of claim 3, wherein an inside diameter of the inner layer is greater than or equal to 0.20 mm and less than or equal to 0.38 mm.

12. The multilayer tube of claim 12, wherein the inside diameter of the inner layer is between 0.26 mm and 0.34 mm, between 0.28 mm and 0.32 mm, or about 0.30 mm.

13. The multilayer tube of claim 3, wherein an inside diameter of the inner layer is greater than or equal to 0.42 mm and less than or equal to 0.58 mm.

14. The multilayer tube of claim 13, wherein the inside diameter of the inner layer is between 0.46 mm and 0.54 mm, between 0.48 mm and 0.52 mm, or about 0.50 mm.

15. The multilayer tube of claim 1, wherein the multilayer tube is configured with a round or an oval cross-section.

16. The multilayer tube of claim 1, wherein the outer layer and the inner layer are joined together by at least one of a form fit, a force fit, a chemical bond, or a shrink fit process.

17. The multilayer tube of claim 1, wherein the outer layer and the inner layer are coextruded.

18. An infusion set for dispensing insulin comprising a multilayer tube configured to receive the insulin, the multilayer tube comprising:
an outer layer comprising polyurethane; and
an inner layer comprising high-density polyethylene and/or polypropylene,
wherein:
an outside diameter of the outer layer is greater than or equal to 1.42 mm and less than or equal to 1.62 mm,
an inside diameter of the outer layer is greater than or equal to 0.50 mm and less than or equal to 0.70 mm,
an outside diameter of the inner layer is greater than or equal to 0.50 mm and less than or equal to 0.70 mm, and
an inside diameter of the inner layer is greater than or equal to 0.20 mm and less than or equal to 0.38 mm.

19. The infusion set of claim 18, wherein the multilayer tube consists of two layers.

20. An infusion set for dispensing insulin comprising a multilayer tube configured to receive the insulin, the multilayer tube comprising:
an outer layer comprising polyurethane; and
an inner layer comprising high-density polyethylene and/or polypropylene,
wherein:
an outside diameter of the outer layer is greater than or equal to 1.42 mm and less than or equal to 1.62 mm,
an inside diameter of the outer layer is greater than or equal to 0.70 mm and less than or equal to 0.90 mm,
an outside diameter of the inner layer is greater than or equal to 0.70 mm and less than or equal to 0.90 mm, and
an inside diameter of the inner layer is greater than or equal to 0.42 mm and less than or equal to 0.58 mm.
